(19) United States
(12) Patent Application Publication
(10) Pub. No.: US 2023/0248300 A1
(43) Pub. Date: Aug. 10, 2023

(54) SYSTEM AND METHOD FOR DETERMINING IMPACT SEVERITY

(71) Applicant: Jasper Medtech Pty Ltd., Sydney (AU)

(72) Inventor: Ralph MOBBS, Sydney (AU)

(21) Appl. No.: 18/135,340

(22) Filed: Apr. 17, 2023

Related U.S. Application Data
(63) Continuation of application No. PCT/AU2023/050034, filed on Jan. 21, 2023.

(30) Foreign Application Priority Data
Jan. 21, 2022 (AU) 2022900121

(51) Int. Cl. A61B 5/00; A61B 5/11

(52) U.S. Cl. CPC A61B 5/4064; A61B 5/1122; A61B 5/1114; A61B 5/682; A61B 5/742; A61B 5/7455; A61B 2562/0219; A61B 2503/10

(57) ABSTRACT

There is provided a system for determining severity of an impact and/or a post impact risk score for a subject, the system comprising an accelerometer configured to output signals indicative of movement of the subject along one or any combination of an x-axis, a y-axis, and a z-axis; a magnetometer configured to output signals indicative of variations in position of the subject in a space defined by the x-axis, the y-axis, and the z-axis; and a gyroscope configured to output signals indicative of angular velocity of the subject around one or any combination of the x-axis, the y-axis, and the z-axis; and a processor configured to receive the output and analyse the output signals to determine for the subject one or any combination of Impact Force, Stun Time, Sway Time, Slow Time and Sway Score, wherein the x-axis is a horizontal axis to the ground directed forward of the subject's body; the y-axis being a horizontal axis to the ground directed laterally of the subject's body; and the z-axis is vertical axis to ground. Also provided are methods to determine a post impact risk score for a subject.

### CROSS REFERENCE TO RELATED APPLICATIONS

This application is a Continuation Application of co-pending International Application Number PCT/AU2023/050034, filed Jan. 21, 2023; which claims the benefit of Australian Provisional Patent Application No. 2022900121, filed on 21 Jan. 2022 the disclosure of which are hereby expressly incorporated by reference in their entirety.

### TECHNICAL FIELD

The present invention relates to a system and method for determining neurological severity of an impact and for assessing a subject's risk of neurological damage following an impact. In particular the impact may be a head or body impact or a consequential movement such as whiplash.

### BACKGROUND

Concussion can be defined as a traumatically induced alteration of mental status representative of neurological dysfunction that may or may not involve loss of consciousness and typically results in impaired mental status, balance, and delayed reaction time, as well as common symptoms (e.g., headache, confusion, diplopia etc). Concussion may correlate to mild traumatic brain injury. To assess potential areas of dysfunction after concussion, clinicians use a variety of testing techniques many of which are highly sensitive acutely post-concussion but demonstrate reduced sensitivity within the week after injury likely owing, in part, to a practice effect from repeat test administration. However, other assessment techniques such as laboratory or instrumented methods of testing are not typically available to the clinician in the field and have identified deficits that persist beyond clinical recovery and self-reported asymptomatic status. In addition, none measure the presence of loss of consciousness at the time of the event, reliant upon the informant. Thus, even if a subject (such as an athlete) can ‘pass’ the routine tests relative to their own baseline performance or normative values, true neurological deficits either may have passed or may continue to exist undetected, and the athlete may return to athletic participation having had, or currently with, residual neurological deficits.

The human brain has a standard response to concussion during the first few seconds after an impact or collision (the Immediate Response). This is in contrast to the heterogeneity of symptoms that become apparent as beyond this time frame. The Immediate Response can comprise complete or partial loss of consciousness, loss of responsivity, loss of coordination, slowness of movement, and changes in heart rate, respiratory rate, sweating and other autonomic responses. If traumatic convulsion is present, trismus or lock-jaw may occur transiently, and this may be represented by a persistent bruxism. These responses, can be measured and taken in to account when assessing whether a subject has suffered neurological damage.

As part of the Immediate Response there may be autonomic changes in heart rate, respiratory rate, sweating may be seen in convulsion due to concussion, and these responses may be measured to determine seizure occurrence. In cases of convulsion due to concussion, posturing, including tonic phase trismus, may be seen and this is currently not measured.

Observational tools for assessing neurological deficits such as concussions typically pertain to biomechanical rather than neurological measurements. In addition, the neurological consequences of a collision are apparent in the period immediately after a collision and measurements are not taken during this period, that is, there are no measures of neurological function during the Immediate Response, accordingly assessment of traumatic brain injury is confounded by the heterogeneity of symptoms that become apparent beyond this time frame of the Immediate Response.

It is well understood that monitoring subjects that have experienced an impact or repeated impacts (for example sportspeople and military personnel) is important to avoid preventable long term complications including post-concussion syndrome, post traumatic migraine, depression, anxiety, post traumatic stress disorder, memory loss, dementia, chronic traumatic encephalopathy, traumatic encephalopathy syndrome, and Parkinsonism.

Monitoring of individual neurological and physiological symptoms does not correlate accurately with recovery and safety to return to an environment where head injury may be sustained. It remains unpredictable who will progress to a more persistent, severe, or fluctuating syndrome post concussion versus those who recover within hours, days or weeks.

At present, monitoring in the community context is performed by on-field or sideline staff, parents and other observers, with minimal or no specific training, using subjective spoken check-list tools. Monitoring in professional settings is performed by medical staff but this assessment is well after the impact.

Accordingly, the present inventors have developed improved systems and methods to provide robust and reliable extraction of impact and post-impact metrics, particularly during the Immediate Response, to provide useful clinical information such as the medium to long term risk of a cognitive impairment and neurological injury or deficit. In addition, real-time, or near real-time measurement of impact metrics for a subject can be used to monitor the severity of an individual impact or the cumulative severity of multiple impacts.

### SUMMARY

In a first aspect there is provided a system for measuring the severity of an impact on a subject, the system comprising:

a) an accelerometer configured to output signals indicative of movement of the subject along one or any combination of an x-axis, a y-axis, and a z-axis

b) a magnetometer configured to output signals indicative of variations in position of the subject in a space defined by the x-axis, the y-axis, and the z-axis; and

c) a gyroscope configured to output signals indicative of angular velocity of the subject around one or any combination of the x-axis, the y-axis, and the z-axis;

d) a processor configured to receive the output signals and analyse the output signals to determine for the subject one or any combination of impact metrics:
i. Impact Force;
ii. Stun Time
iii. Sway Time
iv. Slow Time
v. Sway Score
vi. Trismus

wherein the x-axis is a horizontal axis to the ground directed forward of the subject's body; the y-axis being a horizontal axis to the ground directed laterally of the subject's body; and the z-axis is vertical axis to ground.

The system may further comprise a communications unit for transmitting the output signals and/or one or more the metrics, preferably the Impact Force.

The system may further comprise a display or data analysis unit to receive the transmitted output signals and/or one or more the metrics. For example the transmission of metrics can be in real-time.

In one embodiment the display unit is configured to present a graphical representation of the one or any combination of metrics.

The accelerometer, magnetometer and gyroscope may be in a sensor unit adapted to be disposed on the subject.

The sensor unit may further comprise the processor.

The processor may be configured to determine an Impact and Post Injury Concussion Score (PICS) from at least two of the post impact metrics i-vi.

The PICS may be determined by summing the absolute values, or calculating a weighted average, of two or more of:
i. Impact Force;
ii. Stun Time;
iii. Sway Time;
iv. Slow time; and
v. Sway Score.

In one embodiment the system includes a sensor unit comprising the accelerometer, magnetometer, gyroscope, and optionally the processor.

The sensor unit can be adapted to be disposed on the subject, for example the sensor unit may be associated with a mouthguard, helmet, headgear or apparel.

In one embodiment the mouthguard comprises an indicator to provide a visible or sensory indication of the severity of the impact. For example, the visible indication may be a color change, the sensory indication is vibration or release of a flavorant.

In a second aspect there is provided a method for determining a Post Impact Concussion Score (PICS) for a subject experiencing an impact, the method comprising:

a) determine, for example using the system of the first aspect, for the subject any two or more impact metrics selected from:
i. Impact Force;
ii. Stun Time
iii. Sway Time
iv. Slow Time
v. Sway Score
vi. Trismus

b) assigning a numerical weighting to each impact metric; and

c) summing or calculating a weighted average from the weighted metrics to provide the Post Impact Concussion Score.

The Impact Force may be assigned a weighting of 40%, each of the Stun Time, Sway Time, Slow Time are assigned a combined weighting of 10%, and the Sway Score is assigned a weighting of 50%.

If the Sway Score is above a threshold value (e.g. 0.21-0.60) the numerical weighting assigned to it is not more than 5, 10, 15, or 20 percent of the total of the weightings applied to all the impact metrics.

If the Sway Score is at or below a threshold value (e.g. 0.61-1.2) the numerical weighting assigned to it is 21, 25, 20, 35, 40, 45 or 50 percent of the total of the weightings applied to all the impact metrics.

If the Stun Time is about 10, 15, 20, 25, or 30 seconds or more the Stun Time is weighted as 100 and the other impact metrics as 0.

If the Stun Time is zero, and the Sway Score is 0.21-0.6, the weighting of the Sway score is increased to about 70% and the remaining weightings reduced accordingly.

If the Stun Time is zero, and the Sway Score is 0.61-1.2 then the weighting of the Sway Score is increased to 85% and the remaining weightings reduced accordingly.

If the Sway Score is above 1.2 then the weighting is 100% and the other metrics are weighted as zero.

### DEFINITIONS

As used herein the term ‘PIC’ refers to Post Impact Concussion and the term ‘PICS’ refers to Post Impact Concussion Score.

As used herein the term ‘IMU’ refers to an Inertial Measurement Unit.

The term ‘AP’ refers to Antero-Posterior.

The term ‘ML’ refers to Medio-Lateral.

The term ‘MEMS’ refers to Micro Electro Mechanical Sensors.

Throughout this specification, unless the context clearly requires otherwise, the word ‘comprise’, or variations such as ‘comprises’ or ‘comprising’, will be understood to imply the inclusion of a stated element, integer or step, or group of elements, integers or steps, but not the exclusion of any other element, integer or step, or group of elements, integers or steps. Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (ie, at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

In order that the present technology may be more clearly understood, preferred embodiments will be described with reference to the following drawings and examples.

### DESCRIPTION OF EMBODIMENTS

Concussions (including sport-related concussions) are typically assessed using a variety of testing techniques but it is widely known that even if an athlete can ‘pass’ the routine tests relative to their own baseline performance or normative values, true neurological deficits may often continue to exist.

One such example of a test that may not be able to detect continued physiological/neurological deficits is the Balance Error Scoring System (BESS). It has been well established that impaired balance is one common result of a concussion. The BESS is a frequently used balance test during concussion baseline, diagnosis, and management protocols. Although developed as a feasible test the BESS has a number of limitations, most notably improved performance owing to a practice effect with repeat administration and test environment differences, resulting in poor test metrics. Therefore, reliance on the BESS as a sole indicator of postural control system recovery after concussion or head trauma can result in low sensitivity and potentially clearance to return to athletic activities prior to full neurological recovery. Most athletes achieve baseline BESS values within 3-5 days post-concussion, often despite the continued presence of concussion-related symptoms. While concussion-related symptoms tend to persist for 5-7 days post-concussion, over 75% of athletes achieve baseline BESS values within 48 h, and over 90% demonstrate full BESS recovery within the first week of injury. Thus, the BESS does not provide a comprehensive method to assess postural stability when making return-to-play decisions.

Conversely, instrumented assessments of postural stability such as the Sensory Organization Test (SOT) may be more sensitive to continued deficits in the post-acute phases of concussion. Broadly speaking, the SOT has successfully identified impaired postural control acutely post-concussion with a higher sensitivity than the BESS supporting the notion that instrumented and objective measures of function are a better method to identify post-concussion deficits than clinically used subjective tests. Beyond the composite score outcome variables provided by the SOT. However, use of the SOT in most sporting settings is cost and space prohibitive.

Using force platform technology, several studies have identified deficits beyond clinical recovery and it has been suggested that instrumented measures of postural control are more effective at identifying persistent deficits in postural control than currently used post-concussion assessments (i.e., the BESS). Despite the advantages of such instrumented approaches, these tasks are limited by their reliance on a single task (i.e., only assessing motor function), are static (as opposed to dynamic) in nature, and are not feasible (e.g., cost, expertise) in most environments.

More rigorous approaches to post-concussion deficit detection have continued to show sub-clinical deficits, and the evaluation of neuromuscular control during various tasks, including gait, has allowed for the detection of deficits for a longer period of time after concussion than traditional postural control tests such as the BESS have been able to identify. Gait measurements are a reliable method to quantify postural stability and changes in health status, and the neuromuscular control patterns responsible for gait are developed early in life. Thus, by the time an individual reaches adolescence, they are able to walk with little attentional focus on such an automated motor task.

In is generally accepted that gait and balance evaluations should be included within detailed neurological examinations of concussion. However, instrumented assessments of gait or neuromuscular control are not used in a widespread fashion due to a variety of factors, instrument cost, space, or personnel necessary to operate such protocols. As such, athletes who continue to have subtle deficits that are not detected by traditional clinical neurocognitive and static balance tests may be returned to play under standard clinical guidelines, perhaps prior to full physiological and neurological restoration of the brain. These lingering deficits have the potential to be exacerbated in a more dynamic and cognitively challenging environment, such as during athletic competitions. To maximize goal attainment on the field, an athlete needs to properly distribute their attention across both internal and external stimuli, select appropriate motor responses in response to those stimuli, rapidly implement the response, and monitor and adjust the implementation. Furthermore, these sequences need to be performed while allocating attentional focus among rapidly evolving aspects on the field during play. As with the effects of dual attention tasks in the laboratory environment, the cognitive challenges posed on the field may result in detrimental effects on an athlete's neuromuscular control.

Deficits in neuromuscular control and attention have been linked to an increased risk of musculoskeletal injury independent of concussion. These abilities are not routinely assessed in return-to-play evaluations and residual deficits have been frequently identified using instrumented measures after clinical recovery. Post-concussion neuromuscular and/or attention deficits contribute to an increased risk of musculoskeletal injury upon resumption of athletic activities.

Moreover, neurological deficits associated with a head trauma or collision are not assessed in the period directly following the head trauma or collision.

Human gait is controlled by a complex set of interactions between multiple organ systems. Keeping balance while standing on two feet with relatively small surface area requires complex and delicate interactions between the musculoskeletal system, peripheral nervous system (PNS) and the central nervous system (CNS). The organ systems involved in coordinating gait include muscles with specialized microscopic organelles, including golgi tendon organs that sense tension in the tendons and spindles that sense the speed of muscle stretch. This sensory information is transferred, along with the proprioceptive data carried through the PNS, to different parts of the CNS for a real-time analysis and correction to keep the body in a stable position while moving at different speeds and directions. Keeping the body in a stable position requires the integrity of the CNS where complex back-and-forth interactions between the thalamus, basal ganglia, the sensory-motor cortex, and the cerebellum provide a real-time guidance back to the musculoskeletal system.

Any damage to the structures involved in this process may result in a gait abnormality. As a result, gait abnormalities may present in many different ways. For example head trauma that leads to a concussion may affect an individual's gait and balance before the concussion is developed.

Accordingly, there is provided an improved system and method to measure impact force and abnormal movement patterns in the period immediately following the impact (for example 0.1-30 seconds). The system can capture data (both at a point in time and over time) on a subject's movement and posture including gradual, episodic, and/or random abnormalities and detects and assesses the risk of injury by analyzing those measurements. In some embodiments the system can transmit the data in real-time or near real-time to a display unit or data analysis unit. In turn, the severity of injury or impact (either a single injury/impact or cumulative impacts/injuries) can be used to assess the immediate and progressive risk following an impact.

The system described herein uses non-invasive systems and methods to assess impact and calculate relevant metrics post impact.

The system is useful for assessing the level of immediate and progressive cognitive disability in the context of a head injury and/or concussion.

The systems and methods disclosed herein comprise one or more Inertial Measurement Units (IMUs), commonly known as ‘wearable devices’ or ‘wearables’ which contain various microelectromechanical sensors (MEMS) including accelerometers, gyroscopes and magnetometers. IMUs and are an alternative to the existing methods of head injury assessment. Wearables can accurately measure numerous activity and impact metrics. Accordingly, the systems and methods disclosed herein can be used to monitor the severity of an impact on a subject.

The systems and methods use a sensor device placed on or in a subject's mouthguard, helmet, headgear, apparel or the like. The sensor may be anterior, lateral or posterior. The system includes one or more sensor devices that communicate with a processor that can produce information, based on the sensor readings and data, to facilitate monitoring of the subject by a clinician, doctor, hospital, carer, or other appropriate person.

The system includes a wearable device with one or more sensors, such as accelerometers. For example, the wearable device may include one or more sensors and may form part of the normal sporting equipment used by a subject (e.g., a mouthguard, helmet or headgear). In some embodiments the device is formed into the equipment during manufacture and in other embodiments the device is adapted to be retrofitted to the equipment.

In at least some embodiments, the one or more sensors communicate with a processor and the processor with the sensor/s. The processor may be in the wearable device or may be remote from it. In some embodiments the sensor device also includes a display. In some embodiments, the processor, the sensors, or both communicate with a display device, such as a mobile phone, tablet, or computer.

In one embodiment of a system, the system includes a processor, and one or more sensors in a wearable device.

In at least some embodiments, the one or more sensors and, preferably, the processor (or multiple processors) are provided in a sensor device that is adapted to be applied to the skin of the patient, carried on an article of clothing, carried on a sling or harness, or in a piece of protective equipment (e.g. mouthguard, helmet or headgear) worn by the subject.

Optionally, the system includes a display device. The display device can be any suitable device such as a computer (for example, a notebook or laptop computer, a mobile medical station or computer, a server, a mainframe computer, or a desktop computer), mobile devices (for example, a smartphone, smartwatch, or a tablet), or any other suitable device. In some embodiments, the display device can be incorporated into a medical station or system. In some embodiments the display device comprises a processor for processing and/or displaying information obtained or derived from the sensor(s).

In one embodiment the display unit is is configured to present a graphical representation of the one or any combination of metrics transmitted to it by the system. For example, the display unit may display the Impact Force and/or a Post-Impact Concussion Score (PICS). The Impact Force can be visualized with reference to a scale (e.g., mild, moderate, severe). In this embodiment the data visualization is suitable for non-clinical use and may be used by sports broadcasters as a ‘hit-o-meter’ otherwise known as ‘hit-o’ or ‘hito’ to communicate the severity of an impact to an audience.

In some sports such as boxing and other combat sports the impact metrics can be used to provide an objective score for purpose of victory/success that does not not require ‘subjective’ input from referee or other third party. The sensors may communicate with multiple judges independently. In some embodiments the metrics can be compared to a predetermined threshold value, or a subject's previous data to provide an objective score for purpose of victory/success. That is if one subject is delivering blows to another subject with a force that has previously been found to overcome an opponent, the subject has a high chance of success.

Additionally, or alternatively the display unit can be configured to display other impact metrics such as the Sway Score or Sway Time which can monitored over time to indicate how long the subject takes to regain a normal ambulation pattern after an impact. The Sway Time/Sway Score can be calculated and displayed continuously for a period of time and displayed as a ‘recovery worm’, for example by plotting the Sway Time/Sway Score as a function of time so a viewer can watch a metric of the subject's ambulation pattern after an impact, for example this may be displayed as a score or time and the score may improve as the subject recovers or deteriorate as the subject develops a concussion or other neurological injury.

The system can be used to track and record all impacts and impact metrics for a plurality of subjects (e.g. every player on a field as well as, for examples officials and non-players). The raw data (outputs) and metrics can be recorded to enable the provision of game based metrics for a subject. For example, the system enables the provision of game, season and career impact metrics.

The system can be used to provide one or more of
a post Impact time score with real-time feedback of progression based on metrics of stun/wobble/slow.
a visual representation of Game statistics of impact/s, severity, total-score, etc.
a visual representation of season and life time impact calculation for purpose of entertainment, health status observation, predictive modelling of future impact severtity.

The system can also be used in a method for assessment of impact events ‘off the ball’ that can be assessed both analytically and visually by a third party for either entertainment and/or game-based decision making. Further it can assist in tracking recovery, Monitoring the development of neurological deficit, and/or assessing the risk of impact.

In some embodiments the display device is configured to communicate with one or more other devices and can for example alert a subject's clinician, career or other designated person or service. For example, if the PIC score or other impact metric (see below) indicates that the subject is at a high risk of traumatic brain injury (for example) an alert may be sent to a carer or clinician. Alternatively, if the example if the impact metrics and/or PIC score indicate that the subject is minimally affected by the impact and does not display any clinically relevant signs of traumatic brain injury (for example), this information can be used as an objective measure that the subject is at minimal risk of traumatic brain injury (for example).

In one embodiment of the sensor device, the display device, or both have the ability to process data and comprise a memory, a display, and are adapted to receive an input via an input device. In some embodiments these components can be carried by the user (for example if they are part of the sensor device).

The processor is configured to execute instructions provided to the processor. Such instructions can include any of the steps of methods or processes described herein. Any suitable memory can be used for the sensor and display devices. The memory may be any computer-readable storage media such as, nonvolatile, non-transitory, removable, and non-removable computer-readable media implemented in any method or technology for storage of information, such as computer readable instructions, data structures, program modules, or other data.

Communication methods provide another type of computer readable media, e.g., communication media. Communication media typically embodies computer-readable instructions, data structures, program modules, or other data in a modulated data signal such as a carrier wave, data signal, or other transport mechanism and includes any information delivery media. By way of example, communication media includes wired media such as twisted pair, coaxial cable, fiber optics, wave guides, and other wired media and wireless media such as acoustic, RF, infrared, Bluetooth′, near field communication, and other wireless media.

The display can be any suitable display such as a monitor, screen, display, or the like, and can include a printer.

The input device can be, for example, a keyboard, mouse, touch screen, track ball, joystick, voice recognition system, camera, microphone, or any device known in the art to provide input directly or indirectly to a processor.

The display device may be configured to temporally correlate the data provided by the sensors with video footage of the impact.

In some embodiments the display device is configured to communicate with one or more other devices and can for example alert a subject's clinician, career or other designated person or service. For example, if the impact metrics indicate that the subject is at a high risk of neurological damage, an alert may be sent to a coach or clinician.

A high risk alert may take the form of a visual, auditory, or other sensory alert to the wearer, those around the wearer, or distantly via the display device computer. In some embodiments the high risk alert is communicated to a math official (e.g. a referee or umpire). An alert may be present on the device itself, for example a flashing light, colour change, taste change, vibration, or auditory alert.

Alternatively, if the impact metrics indicate that the subject's movement and posture is not adversely affected by an impact, an alert may be sent to a clinician, electronic medical record, or designated person that the subject ready for re-engagement with a sporting, recreational and/or workplace activity.

In one embodiment of the sensor device, the display device, or both have the ability to process data and comprise a memory, a display, and are adapted to receive an input via an input device. In some embodiments these components can be carried by the user (for example if they are part of the sensor device).

Any suitable type of sensor can be used including, but not limited to, accelerometers, magnetometers, gyroscopes, proximity sensors, infrared sensors, ultrasound sensors, thermistors or other temperature sensors, cameras, piezoelectric or other pressure sensors, sonar sensors, external fluid sensor, skin discoloration sensor, pH sensor, and microphones or any combination thereof.

In at least some embodiments, the system includes at least one, two, three, four, five, six, or more different types of sensors. The system may include at least one, two, three, four, five, six, eight, ten, or more sensors. The sensors may be present in a single sensor device or in multiple sensor devices adapted to be applied to different areas of the subject.

The one or more sensor devices can be used to measure, monitor, or otherwise observe a subjects impact and post-impact movement metrics and therefore their physical/cognitive activity or health; recovery from impact; rehabilitation program, or any combination thereof.

Information sufficient to calculate one or more of the following can be obtained by the sensors: Impact Force, Stun Time, Sway Time, Slow Time, Sway Score.

Other examples of observations or measurements that can be made or interpreted using one or more of the sensors include activity, temperature of skin, pulse or pulse profile or heart rate recovery time after impact or rest duration. The system can observe or measure one or more of these items or any combination of the items.

The sensor device may be adapted to adhere to a mouth guard, clothing, helmet, skin or otherwise be held adjacent to the subject. The sensor device typically includes a housing and an adhesive pad. Alternatively the housing may be adapted to attach to an article of clothing. Within the housing the sensor device comprises one or more sensors, a power source, a communications unit, and optionally a processor. In some embodiments the housing may be at least a portion of a piece of sporting equipment such as a mouthguard, helmet or headgear.

The housing can be made of any suitable material, such as plastic or silicone, and has sufficient flexibility to fit comfortably to, or rest adjacent to the subject's body. In some embodiments the housing is also resistant to water, sweat, and other fluids. In some embodiments the housing is sufficiently water resistant to allow the patient shower, swim, bathe or play sport in the rain while wearing the sensor device.

In some embodiments the sensors, power source, communications unit, and processor are contained within the housing. In some embodiments, a portion of one or more of the sensors, such as a temperature, pulse, or pressure sensor, moisture sensor, or strain gage, may protrude through the housing to allow contact of the sensor or part of the sensor with the patient.

The mouthguard, helmet, headgear, armour, body apparel (or other piece of sporting or military equipment) can include a battery and charging circuit for wireless power delivery to the sensor device and/or the communications unit. Power can be delivered between a transmitter and a receiver located in the device or communications unit by inductive coupling. The charging circuit can be part of a wireless power delivery system configured to supply power to the battery through inductive coupling. The delivered power may then be regulated by the charging circuit for delivery to loads on the sensor device and/or communications unit.

The mouthguard, helmet, headgear, body apparel (or other piece of sporting or military equipment) can support team or group applications. A software application deployed on a tablet (or other computing device) can communicate with and take measurements from a plurality of mouthguards (for example) by pinging each individually to transfer their data to prevent channel crowding. A microcontroller can transfer data to internal memory and/or external memory during acquisition, and then transfer the stored information in response to the ping requesting transfer.

In the case of long distance transmission (for example transmission to a location outside the field of play), the communications unit can either be located in the subjects mouthguard or helmet (or other equipment worn by the subject).

In some embodiments of the sensor device comprises an accelerometer, a gyroscope and a magnetometer. The accelerometer, gyroscope and magnetometer can be used to measure impact and mobility metrics as noted above.

Other suitable sensors include, but are not limited to, a microphone, pulse oximetry sensor, a heart rate monitor, or the like, or any combination thereof. As will be understood, any suitable sensor described above can be included in the sensor unit and any combination of those sensors can be used in the sensor unit.

Power can be provided to the sensors and processor using any suitable power source such as primary cells, coin cell batteries, rechargeable batteries, storage capacitors, other power storage devices, or any combination thereof. In some embodiments, the power is provided by a kinetic energy power to power the components or to or to recharge a battery or other power source coupled to the components. In some embodiments, a wireless power source can be used. In some embodiments the sensor device comprises a charging port for charging the power source. Alternatively, or in addition, wireless charging systems and methods can be used.

All sensors and the processor may be coupled to the same power source or some of the sensors (or even all of the sensors) and sensor processor may have individual power sources.

In some embodiments, the sensors, processor and communications unit are continuously active. In other embodiments, the sensors, processor and communications unit are active intermittently (for example every 0.05, 0.1, 0.5, 1, 5, 10, 15, or 30 seconds). Optionally, the period may be programmable. In one embodiment the period is altered based on data from one or more of the sensors. In another other embodiment the sensors and processor are activated manually or automatically by the sensor device or display device. In some embodiments the sensors and processor are activated automatically when the sensor device is put into motion.

In some embodiments, each sensor may have different activation schedules (e.g. continuous, intermittent, manual). For example, a temperature sensor may measure temperature periodically, a sensor to measure an impact be activated automatically when a G-force in excess of a predetermined threshold is detected, for example in excess of 5 or 10 G.

The processor can be any suitable processor and may include, or be coupled to memory for storing data received from the sensor. The processor can be wired or wirelessly coupled to the sensor. In some embodiments, the processor may include analysis algorithms for analyzing or partially analyzing data received from the sensor. In other embodiments, the processor may be used to receive, store, and transmit data received from the sensors.

The communications unit can be any suitable communications arrangement that can transmit information from the processor or sensors to another device (such as the display device) The communications unit can transmit this information by any suitable wired or wireless technique such as Bluetooth, near field communications, WiFi, infrared, radio frequency, acoustic, optical, or by a wired connection through a data port in the sensor device.

The systems and methods utilise personal characteristics of the subject to assist in determining one or more impact metrics. The personal characteristics can include one or any combination of age, gender, height, weight, level of activity, level of mobility, body mass index (BMI), and leg length discrepancy. In some embodiments, the impact metrics or PIC score may differ based on the subject's gender, age, or height (or any other personal characteristic or combination of personal characteristics).

In at least some embodiments, the ranges for the different impact metrics can be modified for age, gender, height, or other personal characteristics, or any combination thereof.

An application on the display device may provide information regarding the measurement of the impact metrics (for example, lists of the measurements, graphs of the measurements, averages or daily numbers for the measurements or the like or any combination thereof), as well as any of the metrics described above such as the Sway Score. The application may allow a user to access to some or all profile details and may permit access to sensor unit set-up and calibration applications or protocols.

In one embodiment the sensor device is a wearable mouthguard/helmet based sensor which contains a 16 bit 100 Hz triaxial accelerometer for the detection of linear acceleration (anteroposterior, mediolateral, and vertical), a 16 bit 100 Hz triaxial gyroscope for the detection of angular acceleration (pitch, roll and yaw), and a 0.3 μT 25 Hz triaxial magnetometer to assess orientation relative to the Earth's magnetic field (North-South). The data captured by the TEC is stored as a matrix of the values corresponding to each time point (100 captures per second) for up to 30 seconds post-impact.

The sensor device records the entire post impact bout (30 seconds), and the data captured transmitted via Bluetooth™ to a smartphone.

The system is able to calculate the Post Impact Concussion Score (PICS), see below.

A skilled person will be able to create suitable code for data collection from the sensor device, data processing, and outputs for data analysis. The first output from the IMU may be a .html file which documents the vertical acceleration measured by the sensor (y-axis) against time (x-axis) during the impact and post impact time period.

Wearable sensors can sample data at a range of rates. For example, as exemplified herein the sensor at a rate of 100 hz. However, it is envisaged that sample rates from around 20 Hz to 600 Hz, for example suitable sampling rates may be 20, 30, 40, 50, 60, 70, 80, 90, 100, 125, 150, 175, 200, 225, 250, 275, 300, 325, 350, 375, 400, 425, 450, 475, 500, 525, 550, 575, or 600 Hz. Sampling rates in excess of 600 Hz are also compatible with the methods and systems described herein.

### Impact Metrics

As noted above the device can measure various impact metrics including the following:
Impact Force (“Event Detection”) typically measured in G-force/time. This can be G-force associated with acceleration or deceleration.
Stun Time=time the subject is on the ground with minimal or no movement.
Sway Time=time the subject is unsteady or moving with abnormal coordination such that asymmetry is demonstrated across the plane of measurement (e.g. left to right, anterior to posterior, or across the circular axis). Mild Sway Time is defined by the presence of asymmetrical movements of a ratio at least 0.6:0.4 sec for at least 3 seconds, Moderate is defined by the presence of asymmetrical movements of a ratio at least 0.6:0.4 sec for at least 6 seconds, severe is defined by the presence of asymmetrical movements of a ratio at least 0.6:0.4 sec for at least 9 seconds or a fall back to the ground in the seconds following a an impact.
Slow time=time the subject is moving slowly or bradykinesia measure. ‘Moving slowly’ in this context is defined as movement in any direction of <1 m/s or <0.5 m/s.

Other examples of observations or measurements that can be made or interpreted using one or more of the sensors include, skin temperature, pulse, pulse profile, or heart rate recovery time after impact or a period of rest.

The system can observe or measure one or more of these items or any combination of the items.

Impact Force is measured when the subject experiences an event or impact. In this context an impact is a high force or shock applied over a short time period when two or more bodies collide. Such a force may be an acceleration or deceleration and usually has a greater effect than a lower force applied over a proportionally longer period.

In one embodiment Impact Force is measured as the G-force experiences by the subject as a function of time (e.g. G-force/sec or G-force/milli-second).

Table 1 illustrates the G-force experienced by a subject in a range of activities. It is noted that the high G-force that result in a concussion is experience for a short period of time and it is noted that while G-forces associated with an impact of sufficient severity to cause a concussion, these forces are experienced by the subject for a short time only.

TABLE 1
G-forces

| Action | G-force |
|---|---|
| Standing on the ground | 1 G |
| Rollercoaster | 3.5-6.3 G |
| A slap on the back | 4.1 G |
| Formula 1 racing car | 5 G |
| The luge at Whistler | 5.2 G |
| ‘Plopping’ into a chair | 10.1 G |
| Sneezing (open mouth) | 2.9 G |
| Concussion | 80-100 |

Stun Time is the amount of time the subject is on the ground with minimal or no movement. For example when a subject experiences an impact they are often knocked to the ground where they remain motionless or substantially motionless for a period of time. The Stun Time begins when the subject comes to rest on the ground after an impact and ends when the subject regains their feet or when the subject is able to raise their torso greater the 30 degrees relative to the ground, either aided or unaided or when a predetermined maximum time is reached, for example 30 seconds.

Sway Time (time of axis/midline) is the amount of time the subject is unsteady or moving with abnormal coordination. For example when a subject experiences an impact they may attempt to resume play but due to the neurological consequences of the impact their movement will be unsteady or uncoordinated, particularly with reference to a baseline measurement before the impact. For example the subject may stagger or wander aimlessly. The Sway Time begins after the impact and when the unsteady or abnormal movement begins ends when the subject receives aid (for example the physical support of another person), when normal motion resumes, or when a predetermined maximum time is reached.

Slow Time is a measure of bradykinesia and is the amount of time the subject is moving slowly (<1 m/s or <0.5 m/s) after an impact. For example when a subject experiences an impact their movement may be slowed due to the neurological consequences of the impact, particularly with reference to a baseline measurement before the impact. For example the subject's movement patterns (e.g. stride length and directional control) may appear normal but the subject moves more slowly. Sway Time begins after the impact and when the bradykinesia begins, Slow Time ends when the subject receives aid (for example the physical support of another person), or when normal motion resumes.

One measure of the subject's stability and balance post impact (relevant to Sway time) is the Orientation Randomness Metric Score (Sway Score). During motion, the summative motions of individual joints accelerates the subject's head will in three dimensions. The Sway Score measures the ‘wobble’ of the head (as measured from a single-point IMU with that is part of a mouthguard, helmet or headgear (for example). In this embodiment, the sensor device has its x-axis aligned with the initial direction of motion (antero-posterior plane), z-axis aligned with the direction of measured acceleration due to gravity (superior-inferior plane), and y-axis calculated as the cross product of z and x axis (medio-lateral plane).

To describe the motion of the head in three dimensions, the Sway Score measures the ‘wobble’ of the head. In one embodiment, the Sway Score can be calculated from the sensor outputs in the following manner: Let Li be the total length of the path taken by point X (on the sensor device) relative to another fixed point in the horizontal plane during meter i of the subject moving n meters.

$$Sway Score = \frac{{\sum }_{i = 1}^{n} Li}{n} \qquad Equation 1$$

In another embodiment to calculate the Sway Score, first calculate the point pₜ at time step t from the orientation of the body with respect to the world frame, ^{w}Rₜ^{B}. The body orientation, we, is obtained from the orientation measured by the single point IMU, ^{w}Řₜ^{S}, adjusted by a fixed sensor-to-body rotational offset, ^{B}R₀^{S}, as shown in Equation 2. The sensor-to-body offset, ^{B}R₀^{S}, is calculated by assuming an upright pose (i.e., ^{w}R₀^{B}=I_{3×3}) at t=0 as shown in Equation 3. Finally, point pₜ, which is effectively the x and y coordinates of the body z axis with centre at the origin, is calculated using Equation 4.

$${W}^{} {R}_{t}^{B} = {}^{W} {{R}^{⌣}}_{t}^{S} {( {}^{B} {R}_{0}^{S} )}^{T} \qquad Equation 2$$

$${B}^{} {R}_{0}^{S} = {}^{W} {{R}^{⌣}}_{0}^{S} \qquad Equation 3$$

$${p}_{t} = [ \begin{matrix}1 & 0 & 0 \\ 0 & 1 & 0\end{matrix} ] {}^{W} {R}_{t}^{B} [ \begin{matrix}0 \\ 0 \\ 1\end{matrix} ] \qquad Equation 4$$

In some embodiments the Sway Score is used as a standardised method of assessing stability and balance of the subject after an impact.

The risk of a neurological deficit after impact can also be assessed by the objective and quantitative assessment of movement stability provided by the Sway Score or by a combination of impact metrics. Sway Scores for subjects with mild and more serious neurological deficits are defined herein however the intermediate scores between these 2 points provide an indication of the severity of the deficit.

In one embodiment, an Sway Score for a subject of 0.2 or less is indicative of a minimal risk of neurological deficits.

An Sway Score for a subject of 0.21-0.6 is indicative of a low risk of a neurological deficit.

An Sway Score for a patient of 0.61-1.2 indicative of a medium risk of a neurological deficits.

In one embodiment an Sway Score for a patient of more than 1.2 is indicative of a high risk of a neurological deficit.

### Post Impact Concussion Score

Any two or more of the impact metrics (whether measured by the system or not) can be used to calculate a Post Impact Concussion Score (PICS or PIC Score). In some embodiments the PIC Score is calculated by summing the absolute value of each metric and comparing the PIC Score to a threshold PIC Score below which the risk of concussion or neurological damage is minimal.

In some embodiments the PICS is calculated from metrics calculated at a point in time. The point in time may be at impact or within a short period after impact. For example the PICS may calculated from metrics calculated at 0.05, 0.1, 0.5, 1, 5, 10, 15, 30, 60, or 90 seconds after impact, or any other time in this range.

To calculate the PIC Score for a subject, each impact metric is assigned a score out of 100 (or some other number). A score out of 100 is convenient as the score is equivalent to the percentile of the value of the metric relative to a database of healthy control subjects or the subject's own baseline measurements. The PIC Score is the average score of its constituent metrics, or any combination of its constituent metrics. In some embodiments the PIC Score is the weighted average score of its constituent metrics.

In some embodiments each impact metric is assigned a score based on the magnitude of the metric and according to Table 2.

TABLE 2
Metrics and Score

| Impact metric | Magnitude | Score |
|---|---|---|
| Impact Force | 1-15 G | 0 |
| | 16-30 G | 10 |
| | 31-45 G | 25 |
| | 46-60 G | 50 |
| | 61-100 or more G | 100 |
| StunTime | 1-3 seconds | 25 |
| | 3-8 seconds | 50 |
| | 38 or more seconds | 100 |
| Stun Time | 1-10 seconds | 25 |
| | 11-30 seconds | 50 |
| | 31 or more seconds | 100 |
| Sway Time | 1-10 seconds | 25 |
| | 11-30 seconds | 50 |
| | 31 or more seconds | 100 |
| Slow Time | 1-30 seconds | 25 |
| (<0.8 m/s) | 31-60 seconds | 50 |
| | 61 or more seconds | 100 |
| Sway Score | 0.2 or less | 10 |
| | 0.21-0.6 | 20 |
| | 0.61-1.2 | 75 |
| | More than 1.2 | 100 |

If the Sway Score is not in the bottom percentile, then the weightings of each category are shown in Table 3. That is if the Sway Score is above a threshold (e.g. the bottom percentile) the weighting assigned to it, in this embodiment is 10 (i.e. 10% of the combined weightings). Alternatively the weighting assigned to it may be not more than 5, 10, 15, or 20 percent of the total of all the weightings.

TABLE 3
Impact metrics and weighting-low ORM weighting

| Impact metric | Weighting (%) |
|---|---|
| Impact Force | 40 |
| Stun Time | 10 |
| Sway Time | 10 |
| Slow Time | 10 |
| Sway Score | 10 |

However, if the subject's Sway Score is in the bottom percentile, then the weightings are adjusted to reflect the subject's considerable instability, as shown in Table 4. That is if the Sway Score is at or below a threshold (e.g. the bottom percentile) the weighting assigned to it, in this embodiment is 50 (i.e. 50% of the combined weightings). Alternatively, if the Sway Score is at or below a threshold value the weighting assigned to it is 20, 25, 20, 35, 40, 45 or 50 percent of the total of the numerical weightings applied to all the impact categories.

TABLE 4
Impact metrics and weighting-high ORM weighting

| Impact metric | Weighting (%) |
|---|---|
| Impact Force | 40 |
| Stun Time, Sway Time, Slow Time | 10 |
| Sway Score | 50 |

For each metric, lower cut-off values are at the level observed in a clearly concussed subject and below which further deterioration is not clinically meaningful, while upper cut-off values represented the upper limit of ‘normal’ function (above which further improvement is not clinically meaningful).

If the Stun Time is about 10, 15, 20, 25, or 30 seconds or more the Stun Time is weighted as 100 and the other metrics as 0. This can occur when the subject is incapacitated as a result of the impact and is at high risk of developing a concussion or other neurological injury.

In embodiments where the Stun Time is zero, but the Sway Score is 0.21-0.6, or 0.61-1.2, or above 1.2 then the weighting of the Sway Score is increased and the weighting of the other metrics is reduced. For example if the Sway Score is 0.21-0.6 then the weighting is increased by 20% to 70 and the remaining weightings reduced accordingly. In another example if the Sway Score is 0.61-1.2 then the weighting is increased by a further about 20% to 85 and the remaining weightings reduced accordingly. In a further example if the Sway Score is above 1.2 then the weighting is 100 and the other metrics are weighted as zero on the basis that the subject has experienced a clinically significant impact and the impact and is at high risk of developing a concussion or other neurological injury.

It is noted that injuries to the lower limbs or hips that may cause a subject to ambulate abnormally will not typically generate a Sway Score unless there is a concomitant Impact Force of sufficient magnitude to be detected by the system.

In some embodiments the PIC Score is recorded continuously using a wearable device and may be streamed to healthcare providers or other designated persons from any location, allowing for an objective, real-time evaluation of the risk of impact.

1. A system for measuring the severity of an impact on a subject, the system comprising:
a) an accelerometer configured to output signals indicative of movement of the subject along one or any combination of an x-axis, a y-axis, and a z-axis
b) a magnetometer configured to output signals indicative of variations in position of the subject in a space defined by the x-axis, the y-axis, and the z-axis; and
c) a gyroscope configured to output signals indicative of angular velocity of the subject around one or any combination of the x-axis, the y-axis, and the z-axis; and
d) a processor configured to receive the output signals and analyse the output signals to determine for the subject one or any combination of impact metrics:
i) Impact Force;
ii) Stun Time;
iii) Sway Time;
iv) Slow Time;
v) Sway Score; and
vi) Trismus
wherein the x-axis is a horizontal axis to the ground directed forward of the subject's body; the y-axis is a horizontal axis to the ground directed laterally of the subject's body; and
the z-axis is vertical axis to ground.

2. The system of claim 1, further comprising a communications unit for transmitting the output signals and/or one or more the metrics.

3. The system of claim 2, further comprising a display or data analysis unit to receive the transmitted output signals and/or one or more of the metrics.

4. The system of claim 2, wherein the transmission of metrics is in real-time.

5. The system of claim 3, wherein the display unit is configured to present a graphical representation of the one or any combination of metrics.

6. The system of claim 1, wherein the accelerometer, magnetometer and gyroscope are in a sensor unit adapted to be disposed on the subject.

7. The system of claim 6, wherein the sensor unit further comprises the processor.

8. The system of claim 1, wherein the processor is configured to determine an Impact and Post Injury Concussion Score (PICS) from at least two of the post impact metrics i-vi.

9. The system of claim 8, wherein determining the PICS is determined by summing the absolute values, or calculating a weighted average, of two or more of:
i) Impact Force;
ii) Stun Time;
iii) Sway Time;
iv) Slow Time; and
v) Sway Score.

10. The system of claim 1, wherein the system includes a sensor unit comprising the accelerometer, magnetometer, gyroscope, and optionally the processor.

11. The system of claim 6, wherein the sensor unit is associated with a mouthguard, helmet, headgear or apparel.

12. The system of claim 11, wherein the mouthguard comprises an indicator to provide a visible or sensory indication of the severity of the impact.

13. The system of claim 12, wherein the visible indication is a color change.

14. The system of claim 12 wherein the sensory indication is vibration or release of a flavorant.

15. A method for determining a Post Impact Concussion Score (PICS) for a subject experiencing an impact, the method comprising:
a) determining for the subject any two or more impact metrics selected from:
i) Impact Force;
ii) Stun Time;
iii) Sway Time;
iv) Slow Time;
v) Sway Score; and
vi) Trismus
b) assigning a numerical weighting to each impact metric; and
c) summing or calculating a weighted average from the weighted metrics to provide the Post Impact Concussion Score.

16. The method of claim 15, wherein the Impact Force is assigned a weighting of 40%, the Stun Time, Sway Time, Slow Time are assigned a combined weighting of 10%, and the Sway Score is assigned a weighting of 50%.

17. The method of claim 15, wherein if the Sway Score is above a threshold value the numerical weighting assigned to it is not more than 5 percent of the total of the weightings applied to all the impact metrics.

18. The method of claim 17, wherein the threshold value is 0.21-0.60.

19. The method of claim 15, wherein if the Sway Score is at or below a threshold value the numerical weighting assigned to it is 21, 25, 20, 35, 40, 45 or 50 percent of the total of the weightings applied to all the impact metrics.

20. The method of claim 19, wherein the threshold vale is 0.61-1.2

21. The method of claim 15, wherein if the Stun Time is about 10, 15, 20, 25, or 30 seconds or more the Stun Time is weighted as 100 and the other impact metrics as 0.

22. The method of claim 15, wherein if the Stun Time is zero, and the Sway Score is 0.21-0.6, the weighting of the Sway score is increased to about 70% and the remaining weightings reduced accordingly.

23. The method of claim 15, wherein if the Stun Time is zero, and the Sway Score is 0.61-1.2 then the weighting of the Sway Score is increased to 85% and the remaining weightings reduced accordingly.

24. The method of 15, wherein if the Sway Score is above 1.2 then the weighting is 100% and the other metrics are weighted as zero.

25. A method for determining a Post Impact Concussion Score (PICS) for a subject experiencing an impact, the method comprising:
a) determining for the subject any two or more impact metrics selected from:
i) Impact Force;
ii) Stun Time;
iii) Sway Time;
iv) Slow Time;
v) Sway Score; and
vi) Trismus
b) assigning a numerical weighting to each impact metric; and
c) summing or calculating a weighted average from the weighted metrics to provide the Post Impact Concussion Score;
wherein the impact metrics are measured using the system of claim 1.
